(19) United States
(12) Patent Application Publication
(10) Pub. No.: US 2007/0073590 A1
(43) Pub. Date: Mar. 29, 2007

(54) Remote monitor for physiological parameters and durable medical supplies

(76) Inventors: Louis C. Cosentino, Excelsior, MN (US); Daniel L. Cosentino, Chaska, MN (US)

Correspondence Address: MERCHANT & GOULD PC, P.O. BOX 2903, MINNEAPOLIS, MN, 55402-0903, US

(21) Appl. No.: 11/508,516

(22) Filed: Aug. 22, 2006

Related U.S. Application Data
(60) Provisional application No. 60/710,518, filed on Aug. 22, 2005.

(51) Int. Cl. A61B 5/00; G06Q 20/00; G06G 1/14

(52) U.S. Cl. 705/22; 600/365

(57) ABSTRACT

A method and system for determining need for additional medical supplies includes receiving a test result from a remote computing device. The method and system also include updating a supply counter based on receiving the test result and determining if the supply counter exceeds a limit. The method and system further include triggering a process to reorder supplies when the supply counter exceeds the limit. A system for remote physiological parameter monitoring is also disclosed, and includes a remote computing system and a local computing system. The remote computing system tests the physiological parameter of the ambulatory patient. The local computing system receives the physiological parameter from the remote computing system through a communication network. The local computing system tracks the physiological parameter of the ambulatory patient, and if the physiological parameter is outside certain parameters, the local computing system alerts a caregiver such that the caregiver can contact the ambulatory patient.

### CROSS REFERENCE TO RELATED APPLICATIONS

This application claims priority under 35 U.S.C. § 119(e) to U.S. provisional application Ser. No. 60/710,518, filed Aug. 22, 2005 and entitled “Apparatus and Method For Determining if Patient Needs Additional Medical Supplies”. The entire disclosure of 60/710,518 is incorporated herein by reference.

### TECHNICAL FIELD

The present invention relates to medical monitoring equipment. More specifically, the invention relates to remote monitoring of patient health and patient testing supplies.

### BACKGROUND

Millions of people require durable medical equipment supplies on a regular basis. For example, patients with diabetes must control their blood sugar or glucose. Most people with diabetes use glucose meters, or glucometers, to check their blood sugar. To test for glucose with a typical glucose meter, a small amount of blood is placed on a disposable test strip and placed in the meter. The test strips are coated with chemicals (glucose oxidase, dehydrogenase, or hexokinase) that combine with glucose in the blood. The meter measures how much glucose is present.

Other chronic diseases, such as heart disease, require in-home monitoring of symptoms such as cholesterol. Such monitoring requires semi-regular usage of durable medical supplies as well. For example, a patient may need to take a cholesterol test periodically to allow a caregiver to closely monitor the person's health status. Although at-home cholesterol test kits are available, each cholesterol test generally occurs during a visit to a clinic or hospital, requiring direct caregiver attention.

Because patients require such single-use durable medical equipment supplies on a regular basis, they must constantly monitor their supplies. Patients must then reorder supplies on their own when needed. For example, a patient with diabetes might use 3 test strips per day or close to 100 per month. If test strips are packaged in groups of 100, a patient must reorder supplies on a monthly basis.

Regular contact with patients is often desirable, as allowing medical professional caregivers to monitor and manage a patient's condition reduces hospitalizations by early identification of symptoms, prevents unnecessary hospitalizations and office visits, and provides immediate feedback of a patient's status thus allowing medication and fluid adjustments to be made over the telephone as necessary. Such contact can be made in person; however, managing patients in person is expensive, because regular preventative and monitoring contact takes up a large portion of a medical caregiver's time.

For the foregoing reasons, it is evident that there exists a need for a system that addresses the above described needs in a simple-to-operate and cost effective manner to manage large patient populations.

### SUMMARY

The present invention is directed to a method and system for determining need for additional medical supplies. The method includes receiving a test result from a remote computing device. The method also includes updating a supply counter based on receiving the test result. The method also includes determining if the supply counter exceeds a limit. The method further includes triggering a process to reorder supplies when the supply counter exceeds the limit.

The test results received from the remote computing device could be from a blood glucose level test, a cholesterol test, or any other test using similarly disposable, single-use durable medical supplies.

The supply counter, in various embodiments of the invention, updates and stores the number of test results received such that the method and system described know how many tests have occurred since supplies were last ordered. This updating can be accomplished through use of an up-counter, down-counter, or up-down counter depending on a starting value and selected limit.

The automatic triggering occurs when the supply counter exceeds the limit. By exceeds, it is understood that the supply counter can count up or down toward a selected limit value from a set starting value.

The present invention is also directed to a system for remote physiological parameter monitoring. The system includes a remote computing system and a local computing system. The remote computing system tests the physiological parameter of the ambulatory patient. A physiological parameter, for example, can be a blood glucose level or cholesterol level, but is intended to encompass any and all health test results capable of communication to a local system. The remote computing system also includes a communication device connected to a communication network. The local computing system includes a communication device connected to the communication network. The local computing system receives the physiological parameter from the remote computing system through the communication network. The local computing system tracks the physiological parameter of the ambulatory patient, and if the physiological parameter is outside certain parameters, the local computing system alerts a caregiver such that the caregiver can contact the ambulatory patient.

### BRIEF DESCRIPTION OF THE DRAWINGS

FIG. 1 is a flowchart of a system for determining if a patient needs additional medical supplies;

FIG. 2 is a block diagram of a system for remotely monitoring physiological parameters;

FIG. 3 is a block diagram of a system for remote physiological parameter monitoring;

FIG. 4 is a block diagram of a local computing system for remote physiological parameter monitoring according to a possible embodiment;

FIG. 5 is a block diagram of a remote computing system according to a possible embodiment;

FIG. 6 is a block diagram of a remote computing system according to another possible embodiment;

FIG. 7 is a block diagram of a remote computing system according to another possible embodiment;

FIG. 8 is a flowchart for usage of a remote computing system according to a possible embodiment;

FIGS. 9A-9E illustrate several embodiments of the structure of the remote computing system;

FIG. 10 illustrates the structure of a remote computing system with a support member in accordance with a possible embodiment;

FIG. 11 illustrates the structure of a remote computing system with a support member in accordance with a possible embodiment;

FIG. 12 illustrates a sectional view of an electronic scale in accordance with a possible embodiment of the invention; and

FIG. 13 illustrates a top plate of the electronic scale in accordance with a possible embodiment.

### DETAILED DESCRIPTION

In general terms the present disclosure relates to monitoring or measuring physiological parameters, such as a patient's glucose level, through a remote apparatus. In addition, the need to reorder single-use medical supplies can be determined. For example, each time a patient's glucose level is measured, the patient used a test strip and inserted it into the apparatus for measuring. Such insertion necessarily indicates that the patient has used a test strip. After a number of insertions of the test strip, it can be determined that the patient is running low on test strips.

The embodiments described herein are preferably implemented as a medical apparatus, system and method capable of monitoring wellness parameters and physiological data of ambulatory patients and transmitting such parameters and data from the monitoring device residing at a remote location to a local location. At the local location a medical professional caregiver or logic system can remotely monitor the patient's condition and provide medical treatment as may be necessary.

Preferably, the remote computing system incorporates transducing devices for converting the desired measured parameters into electrical signals capable of being processed by a computing system or microprocessor system. The device remotely interacts with the ambulatory patient and then transmits the measured parameters to a computer located at a local site. At the local location the various indicia of the ambulatory patient's condition are monitored and analyzed. To provide the ambulatory patient with an added level of convenience and ease of use, such monitoring device can be contained in a single integrated package. Communication is established between the remote monitoring apparatus and a local computer via a modem or other electronic communication devices that are generally well known commercially available products. At the local location, the patient's condition is analyzed based on the information communicated (e.g. wellness parameters and physiological data) and can provoke medical treatment in accordance with such information.

Patients suffering from chronic diseases, such as diabetes, can undergo drug therapy and lifestyle changes to manage their medical condition. In such patients, the medical professional caregiver monitors certain physiological parameters such as blood glucose level. Patients will also benefit from daily reminders to take medications (improving compliance) and/or perform some type of exercise. With the information received from the monitoring device, the medical professional caregiver can track the patient's test history and determine the effectiveness of any drug therapy, the patient's condition, whether the patient's condition is improving or whether the patient requires hospitalization or an office consultation to prevent the condition from getting worse.

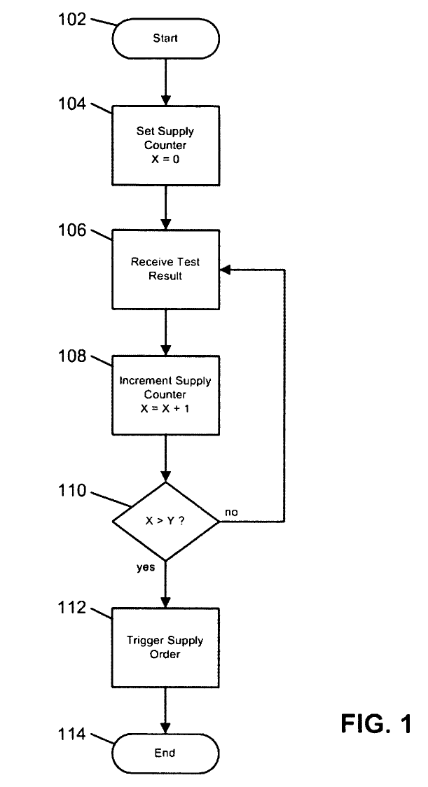

Referring now to FIG. 1, a flowchart of a system 100 for determining if a patient needs additional medical supplies is shown according to a possible aspect of the present disclosure. The logical flow begins at start point 102. A set module 104 sets the supply counter X to an initial value. In the embodiment as shown, the supply counter X is set to equal zero. A receive module 106 receives a test result. An update module 108 increments or decrements the supply counter toward a preset limit Y, depending on the particular implementation of the counter and module. For example, the counter could count up toward a preset “ceiling” value, or count down toward a preset “floor” value.

A determination operation 110 determines if X has exceeded the limit set by Y. Y can be the predetermined level at which reordering takes place. If the determination operation 110 determines that X has not exceeded Y, then logical flow branches “NO” to the receive module 106. If the determination operation 110 determines that X has exceeded Y, then logical flow branches “YES” to a trigger module 112. The trigger module 112 triggers reordering the supplies. Logical flow ends at 114.

The trigger module 112 could automatically order supplies, have them shipped to the patient, and bill the patient's account for such service. Alternatively, the trigger module 112 could prompt the user to confirm that the user wishes to reorder the supplies. This might ensure that the patient actually needs additional supplies. It is possible that the trigger system could reverse the counter X by some amount, for example, 10 and then after 10 more test results are received the trigger module 112 would prompt the patient again. Other alternative arrangements could also be used.

The logical flow of FIG. 1 can best be understood by an application example. Using the example of a patient with diabetes, logical flow begins at start point 102. The set module sets the test strip counter X to zero test strips. The receive module 106 receives a glucose test from the apparatus 10 indicating that the patient has used a test strip. The update module 108 increments the test strip counter to 1. If Y equals 75, the determination operation 110 determines that 1 is not greater than 75, and operational flow branches “NO” to the receive module 106. This process continues until the 76th test result is received by the receive module 106. The update module 108 would set X to 76. The determination operation 110 determines that 76 is greater than 75, and operational flow branches “YES” to the trigger module 112. The trigger module 112 triggers a supply order and operational flow ends at 114. It is noted that this process could repeat indefinitely. Each time the logical flow repeats, the set module 104 would reset X to zero. Alternately, upon ordering a given number of supplies that given number can be added to Y, which would then represent a total number of tests completed.

The logic described above could be used for any supply ordering and reordering using the example descriptions described herein.

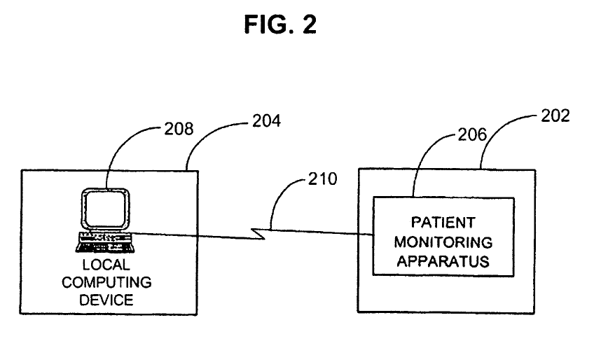

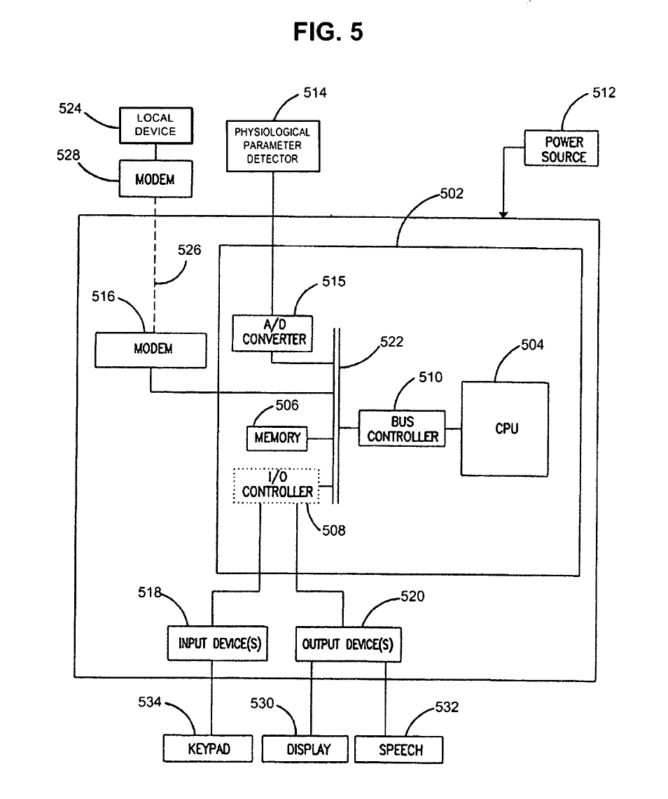

Referring now to FIG. 2, a block diagram of a system 200 for remote physiological parameter monitoring is shown according to a possible embodiment. System 200 incorporates a remote site 202 and a local site 204. The remote site 202 includes a remote computing system, such as remote monitoring device 206. The remote computing system is described in more detail in conjunction with FIGS. 5-7 below. The local site 204 includes a local computing system 208.

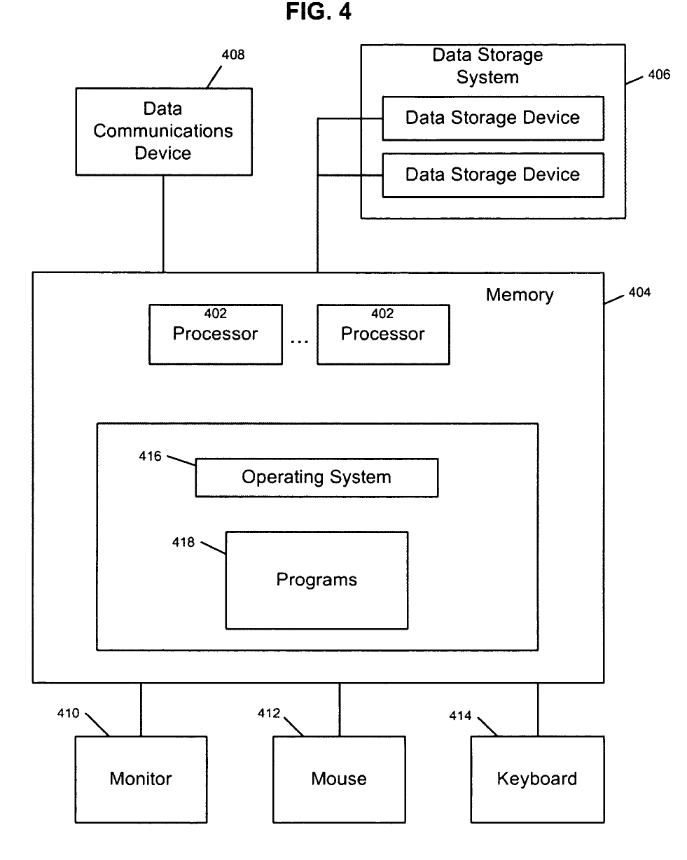

The local computing system 208 can be the system that performs the operations and/or contains the modules associated with FIG. 1. The local computer system can be any of a number of different computing systems, one such embodiment described below in conjunction with FIG. 4. The local computing system 208 and remote computing system, such as remote monitoring device 206, are operatively connected by a communication network 210, the communication network 210 being any type of communication network such as the telephone network, wide area network or Internet.

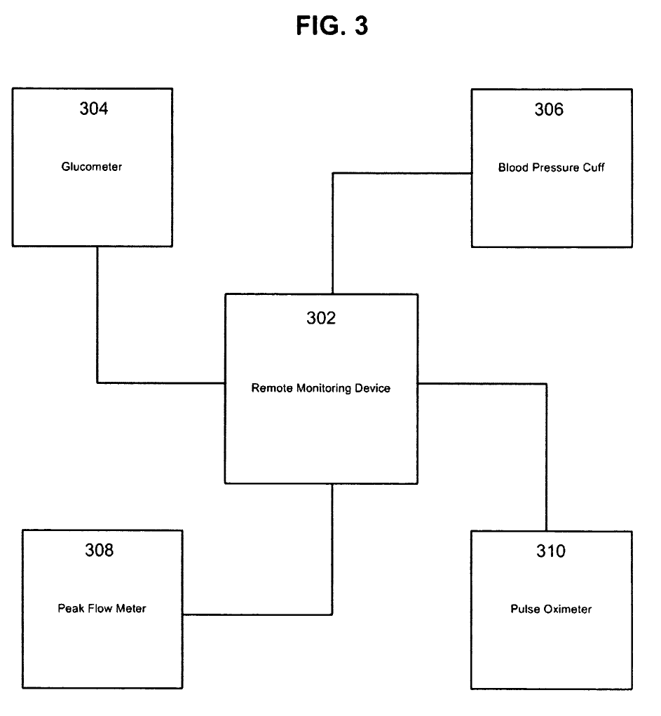

Referring now to FIG. 3, a block diagram of a remote computing system 300 for measuring physiological parameters is shown according to a possible embodiment of the present disclosure. The remote computing system 300 includes a remote monitoring device 302. The remote monitoring device 302 can be any of a number of communicative monitoring devices, examples of which can be seen in FIGS. 5-7. The system also has a variety of peripheral devices for measurement of physical parameters. In the embodiment shown, a glucometer 304, a blood pressure cuff 306, a peak flow meter 308, and a pulse oximeter 310 are operatively connected to the remote monitoring device. Either the peripheral device or the remote monitoring device 302 have the ability to transduce the physiological parameter as measured into an electrical signal for communication to a local computing system as described below.

In one embodiment of the disclosure, namely for diabetic patients, the physiological parameter monitored is the patient's blood glucose level. However, it will be appreciated by those skilled in the art that the physiological parameters can include blood pressure, EKG, temperature, urine output, and any other. Further, the weight of a patient can be measured, as described in the embodiments below.

One or more of the peripheral devices 304-310 can be operatively disconnected from the remote monitoring device 302 either by unplugging a cable or disabling wireless communications. If a given device is functional while detached from the remote monitoring device 302, it stores the measurements of the given physiological parameter from a given test and transmits it to the remote monitoring device 302 when reconnected by attachment of a cable or enabling of a wireless communications conduit.

Similar to that discussed above, glucose levels of a patient with diabetes can be monitored. The patient can insert a test strip, having a small amount of blood, into the glucometer 304. The glucometer 304 can measure the glucose level in the blood and transmit that information through a communication device incorporated into the remote monitoring device 302. The glucose level can be transmitted over a communication network such as the one discussed in conjunction with FIG. 2 to a local computing device. The local computing device can store, track, and monitor the glucose levels of the patient. If the glucose level is abnormal, a caregiver can be notified.

Because diabetic patients generally test blood glucose levels more than once daily, a caregiver has at least daily access to blood glucose test results by use of such a system. This allows the caregiver to intervene sooner and prevent development of serious health issues than would be possible with only medical office or clinic visits.

Now referring to FIG. 4, a diagram of a local computing system 400 for monitoring of physiological parameters is shown according to a possible embodiment. In this embodiment, a local computer system 400 is located at a distance from a remote computing system, such as the one shown in FIGS. 5-7. The local computer system 400 can be used to enter and update a medical professional caregiver's (e.g., a physician) and a patient's records; monitor patient status; issue exception reports; and issue trend reports. The local computing system generally includes one or more processors 402, random access memory (RAM) 404, a data storage system 406 including one or more data storage devices (e.g., hard, floppy and/or CD-ROM disk drives, etc.), data communications devices 408 (e.g., modems, network interfaces, etc.), monitor 410 (e.g., CRT, LCD display, etc.), mouse pointing device 412 and keyboard 414. It is envisioned that the local computing system 400 can be interfaced with other devices, such as read-only memory (ROM), video card, bus interface, speakers, printers, or any other device adapted and configured to interface with the local computing system 400 that is capable of providing an output from the system. Those skilled in the art will recognize that any combination of the above components or any number of different components, peripherals and other devices can be used with the computing system. For example, the system 400 can include an 8 channel MODEM; CD-ROM Back-up: CD-ReWritable, CD-Recordable Drive; and a 17 inch monitor. Those skilled in the art will also appreciate that remote computing devices, such as those described above in conjunction with FIGS. 5-7, will generally have a similar hardware implementation as the local computing system and will be able to communicate with it according to a common interface.

The local computing system can include one or more data communications devices 408 allowing it to communicatively connect to multiple remote monitoring devices, such as the remote computing systems discussed in conjunction with FIGS. 5-7. For example, the local computing system can be provided with a multi-channel modem that allows connection to multiple remote monitoring devices for purposes of downloading physiological parameter information. In one embodiment, a local computing system 400 can be provided with an 8 channel MODEM that allows up to eight patient remote computing systems to simultaneously access and transmit physiological parameter information to the local computing system.

In one embodiment of the present disclosure, the CD-ROM Back-up: CD-ReWritable, CD-Recordable Drive automatically stores a duplicate (back-up) copy of all patient and medical professional caregiver (e.g., physician) data on a compact disc (CD) each night. The CD can store approximately one year of patient data. A new CD should be installed each year. The used CD should be labeled and stored for future reference. In accordance with the principles of this disclosure, a database of patient and medical professional caregiver (e.g., physician) data is updated, maintained and managed by the central computer system.

The local computing system 400 can include a local operating system 416 and one or more programs 418 resident in local memory 404 or on data storage devices 406. This software can facilitate the storage of received physiological parameter information from the remote computing systems as measured by, for example, peripheral devices described in conjunction with FIG. 3.

Because certain physiological parameters require testing using single-use medical equipment supplies, the amount of supplies on hand by the patient can be tracked by the local computing system 400. For example, in the above example of the patient with diabetes, each time the glucose level is transmitted to the local computing system, the local computing system 400 can track that one test strip has been used. After a certain number of transmissions of the blood glucose test, the local computing system 400 can order new supplies for the patient.

For example, if the patient begins with 100 test strips, after approximately 75 tests have been transmitted to the local computing system 400, the local computing system 400 can order another 100 test strips to be sent to the patient. As such, the reordering of the medical supplies can become automated such that the patient does not run out of supplies. This can be accomplished using the method and system described herein. Such an automated system and method is convenient for the patient, as it alleviates the need for the patient to monitor his supply level. The ordering process can be automated along with the billing for such supplies.

Furthermore, a health professional or other caregiver can use the system 400 to readily determine the regularity with which patients are testing their physiological parameters. By examining stored records, a caregiver may choose to contact a patient to encourage more or less testing as appropriate. Alternately, the local computing system 400 could create an alert for the caregiver pointing out the abnormality in testing procedures. Further, the system 400 could send a message directly to the remote computing system such that the patient is notified of a need to alter their testing habits or procedure without the need for caregiver intervention. To aid in illustrating such functionality, the following example is instructive.

Continuing with the example of blood glucose tests, a caregiver has a month of stored glucose testing results on the local computing device. The caregiver sees that the patient has only 15 test results, or sees that a new order of test strips has not been placed in an abnormally long period of time. The caregiver can contact the patient, or the local computing system can be set to contact the caregiver and/or patient once a certain testing regularity is not followed.

Referring now to FIG. 5, a block diagram of a remote computing system 500 for remote physiological parameter monitoring is shown according to a possible embodiment. The system 500 includes microprocessor system 502 including a CPU 504, a memory 506, an optional input/output (I/O) controller 508 and a bus controller 510 as illustrated. It will be appreciated that the microprocessor system 502 is available in a wide variety of configurations and is based on CPU chips such as the Intel, Motorola or Microchip PIC family of microprocessors or microcontrollers.

It will be appreciated by those skilled in the art that the remote computing system requires an electrical power source 512 to operate. As such, the remote computing system can be powered by: ordinary household A/C line power, DC batteries or rechargeable batteries. Power source 512 provides electrical power to the housing for operating the electronic devices. A power source 512 for operating a physiological parameter detector 514 is generated within the housing, however those skilled in the art will recognize that a separate power supply can be provided or the power source 512 can be adapted to provide the proper voltage or current for operating the detector 514.

The remote computing system 500 includes a microprocessor system 502, operatively connected to an electronic receiver/transmitter communication device such as a modem 516, an input device 518 and an output device 520. The modem 516 is operatively coupled to the microprocessor system 502 via the electronic bus 522, and to a local computing system 524 via a communication network 526 and modem 528. The communication network 526 can be any communication network such as the telephone network, wide area network or Internet. It will be appreciated that the modem 516 is a generally well known commercially available product available in a variety of configurations operating at a variety of BAUD rates. In one embodiment of the present disclosure the modem 516 is asynchronous, operates at 2400 BAUD or higher and is readily available off-the-shelf from companies such as Rockwell or Silicon Systems Inc. (SSI).

The physiological parameter detector 514 can measure any of a wide range of physiological parameters including blood glucose level, cholesterol level, lung capacity, heart rate, or weight. One or more such physiological detectors 514 can be interfaced to the system, such as a glucometer, scale, or other detector. If the detector 514 produces a transduced analog signal, an analog-to-digital converter 515 can be used to translate the signal to a digital signal recognizable by the bus controller 510 and processing unit 504 such that it can be transmitted on the communication network 526 via the modem 516.

It will be appreciated that output device(s) 520 can be interfaced with the microprocessor system 502. These output devices 520 include a visual electronic display device 530 and/or a synthetic speech device 532. Electronic display devices 530 are well known in the art and are available in a variety of technologies such as vacuum fluorescent, liquid crystal or Light Emitting Diode (LED). The patient reads alphanumeric data as it scrolls on the electronic display device 530. Output devices 520 include a synthetic speech output device 532 such as a Chipcorder manufactured by ISD (part No. 4003). Still, other output devices 520 include pacemaker data input devices, drug infusion pumps or transformer coupled transmitters.

It will be appreciated that input device(s) 518 can also be interfaced with the microprocessor system 502. In one embodiment of the present disclosure an electronic keypad 534 is provided for the patient to enter responses into the remote computing system 500. Patient data entered through the electronic keypad 534 can be scrolled on the electronic display 530 or played back on the synthetic speech device 532.

In alternate embodiments the input device can include a generic speech recognition device such as those made by International Business Machines (IBM), Dragon Systems, Inc. and other providers. Accordingly, the patient replies to the interrogations merely by speaking either “YES” or “NO” responses into the speech recognition input device.

The microprocessor system 502 is operatively coupled to the modem 516, the input device(s) 518 and the output device(s) 520. The physiological parameter detector 514 is operatively coupled to the microprocessor system 502. Electronic measurement signals from the detector 514 are processed by the A/D converter 515. This digitized representation of the measured signal is then interfaced to the CPU 514 via the electronic bus 522 and the bus controller 510. In one embodiment of the present disclosure, the physiological transducing device includes the physiological parameter detector 514.

Using the input devices 518, output devices 520, and modem 516, the system 500 can be used to allow patients to communicate directly with other computing devices, for example a local computing device as described in conjunction with FIG. 4. Specifically, a caregiver using the local computing device can send queries to the remote computing system 800 through the communication network 526. Alternately, the local computing system can send predetermined messages to the remote computing system 500 and responses logged on the local computing device.

A patient using the remote computing system 800 can view or hear these messages using output devices 520 and respond to them using input devices 518. Such messages can include providing instructions for monitoring physiological parameters, reporting symptoms, or other messages such as those directed toward testing regularity as described below.

It will be appreciated that Analog-to-Digital (A/D) converters are also generally well known and commercially available in a variety of configurations. Furthermore, an A/D converter 515 can be included within the physiological transducing device or within the microprocessor system 502 or within the remote computing system 500 generally. One skilled in the art would have a variety of design choices in interfacing a transducing device comprising an electronic sensor or transducer with the microprocessor system 502.

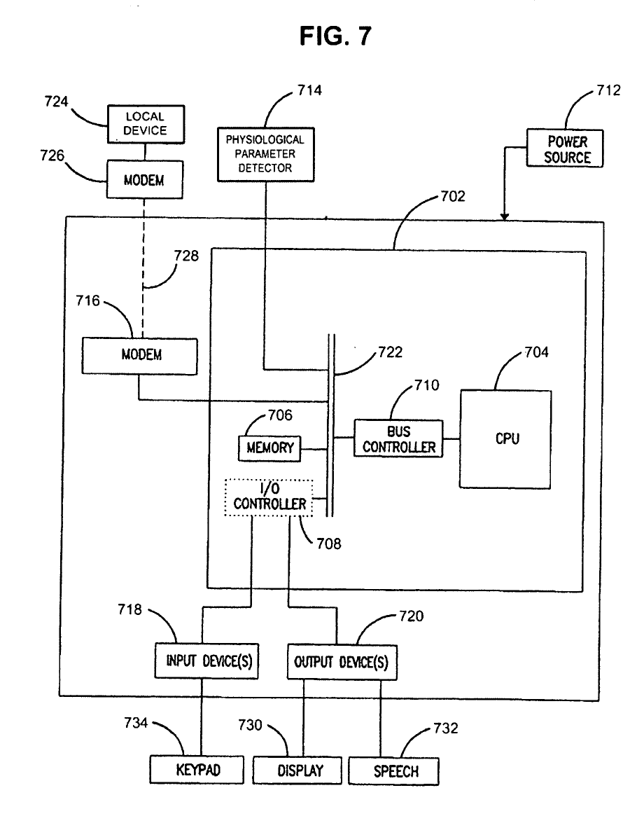

The physiological parameter detector 514 can provide an analog or digital electronic signal output depending on the particular type of detector 514 chosen. If the physiological parameter detector 514 provides an analog output signal in response to a weight input, the analog signal is converted to a digital signal via the A/D converter 515. The digital signal is then interfaced with the electronic bus 522 and the CPU 504. If the physiological parameter detector 514 provides a digital output signal, the digital signal can be interfaced directly with electronic bus 522 and the CPU 504, such as is shown in FIG. 7.

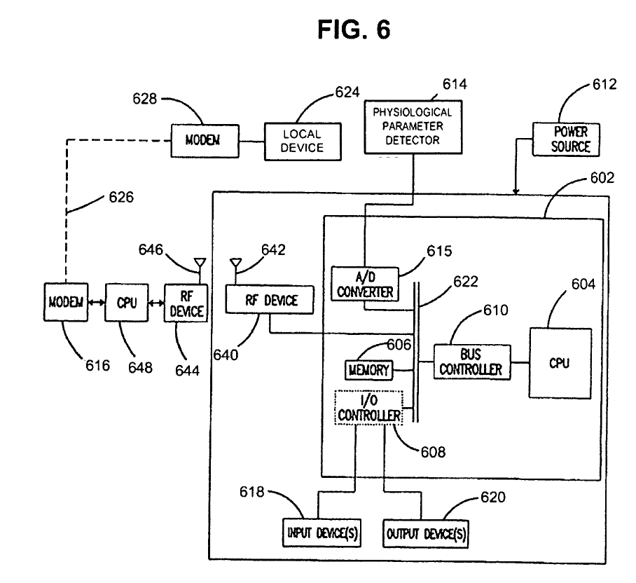

Referring now to FIG. 6, a block diagram of a remote computing system 600 for remote physiological parameter monitoring is shown according to a possible embodiment. The remote computing system 600 includes microprocessor system 602 including a CPU 604, a memory 606, an optional input/output (I/O) controller 608 and a bus controller 610 as illustrated. These components can be configured similarly to those described above in FIG. 5.

The remote computing system 600 also includes a microprocessor system 602, operatively connected to an electronic receiver/transmitter communication device such as a modem 616, an input device 618 and an output device 620. The modem 616 is operatively coupled to the microprocessor system 602 via the electronic bus 622, and to a local computing system 624 via a communication network 626 and modem 628. The physiological parameter detector 614 is operatively coupled to the microprocessor unit 602. Electronic measurement signals from the detector 614 are processed by the A/D converter 615, as discussed above.

In this embodiment, the communication device is a radio frequency (RF) transceiver. The transceiver comprises a first radio frequency device 640 including an antenna 642, and a second radio frequency device 644, including an antenna 646. The first radio frequency device 640 is operatively coupled to the microprocessor system 602 via the electronic bus 622, and is in radio communication with the second radio frequency device 644. The second radio frequency device 644 is operatively coupled through a microprocessor 648 that is operatively coupled to a modem 616. The modem 616 is coupled to the communication network 626 and is in communication with the local computing system 624 via the modem 616. The first radio frequency device 640 and the second radio frequency device 644 are remotely located, one from the other. It will be appreciated that such radio frequency devices 640, 644 are generally well known and are commercially available products from RF Monolithics Inc. (RFM).

In one embodiment of the present disclosure, such transceivers operate at radio frequencies in the range of 900-2400 MHz. Information from the microprocessor system 602 is encoded and modulated by the first RF device 640 for subsequent transmission to the second RF device 644, located remotely therefrom. The second RF device 644 is coupled to a conventional modem 616 via the microprocessor 648. The modem 616 is coupled to the communication network 626 via an in-house wiring connection and ultimately to the modem 628 coupled to the local computing system 624. Accordingly, information can be transmitted to and from the microprocessor system 602 via the RF devices 640, 644 via a radio wave or radio frequency link, thus providing added portability and flexibility remote computing system 600. It will be appreciated that various other communications devices can be utilized such as RS-232 serial communication connections, Internet communications connection as well as satellite communication connections. Other communications devices that operate by transmitting and receiving infra-red (IR) energy can be utilized to provide a wireless communication link between the remote computing system 600 and a conveniently located network connection. Furthermore, X-10 type devices can also be used as part of a communication link between the remote computing system 600 and a convenient network connection in the home. X-10 USA and other companies manufacture a variety of devices that transmit/receive data without the need for any special wiring. The devices works by sending signals through the home's regular electrical wires using what is called power line carrier (PLC).

Referring now to FIG. 7, a block diagram of a remote computing system 700 for remote physiological parameter monitoring is shown according to a possible embodiment. The system 700 includes microprocessor system 702 including a CPU 704, a memory 706, an optional input/output (I/O) controller 708 and a bus controller 710 as illustrated. These components can be configured similarly to those described above in FIGS. 5-6.

The remote computing system 700 also includes a microprocessor system 702, operatively connected to an electronic receiver/transmitter communication device such as a modem 716, an input device 718 and an output device 720. The modem 716 is operatively coupled to the microprocessor system 702 via the electronic bus 722, and to a local computing system 724 via a communication network 726 and modem 728. The physiological parameter detector 714 is operatively coupled to the microprocessor unit 702.

In this embodiment, a digital physiological parameter detector 750 is provided. Digital weight measurements from the digital physiological parameter detector 750 can be interfaced with the microprocessor system 702 and CPU 704 without requiring additional amplification, signal conditioning and A/D converters.

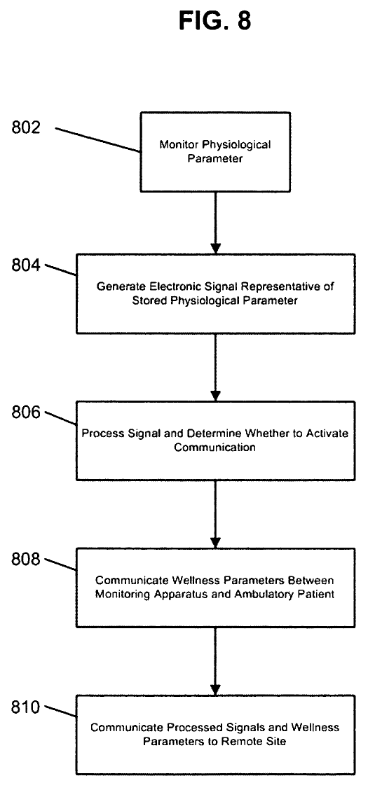

Referring now to FIG. 8, a flowchart for usage of a remote computing system 800 for remote physiological parameter monitoring is shown according to a possible embodiment. A monitor module 802 measures an ambulatory patient's physiological parameter. In one embodiment of the disclosure, namely for diabetics, the physiological parameter monitored is the patient's blood glucose level. However, it will be appreciated by those skilled in the art that the physiological parameters can include blood pressure, lung capacity, EKG, temperature, urine output and any other such physical parameter.

Transduction module 804 converts a monitored or measured physiological parameter from a mechanical input to an electronic output by utilizing a transducing device. In one embodiment of the present disclosure, the transducing device is a glucometer such as the one disclosed in FIG. 3, which converts the patient's blood glucose level into a useable electronic signal.

It will be appreciated that other physiological transducing devices can be utilized in addition to or alternately to the glucometer. For example, a blood pressure measurement apparatus and an electrocardiogram (EKG) measurement apparatus can be utilized for recordation and/or transmission of blood pressure and EKG measurements from a remote location. An electronic scale can be utilized for measuring and monitoring weight changes. It will be appreciated that other monitoring devices of physiological body functions that provide an analog or digital electronic output can be utilized, as described with various embodiments of a remote computing system as described in FIGS. 5-7.

Processing module 806 processes the electronic signal representative of the transduced physiological parameter. In some embodiments, the processing module 806 can determine whether the resulting parameter value is within certain preprogrammed limits. If so the remote computing system 800 initiates communication within a local computer (such as the one shown in FIG. 2) via a communication device and over a communication network.

User communication module 808 communicates physiological parameters between the remote computing system 800 and the ambulatory patient. For example, the results of a measurement of a physiological parameter, such as a blood glucose level, can be communicated to the patient.

Remote communication module 810 communicates physiological parameters between the remote computing system 800 and a local computing system, such as the one shown in FIG. 2.

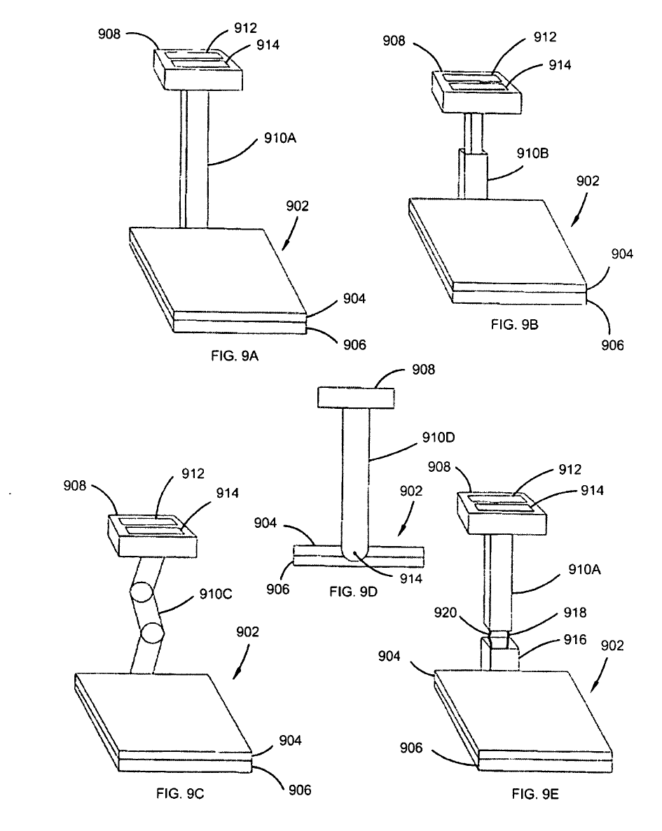

Referring now to FIGS. 9-13, a variety of possible structural embodiments of the remote computing system as described above are shown according to the present disclosure. In such embodiments, the remote computing system as described above takes the form of a specialized patient monitoring apparatus including a rarity of monitoring systems for measuring one or more physiological parameters such as blood sugar levels or weight.

Referring now to FIG. 9A, as this embodiment of the present disclosure is described herein, an integrated remote computing system 900 is shown. Preferably, the remote computing system 900 includes an electronic scale 902. The electronic scale 902 further includes a top plate 904 and a base plate 906. The remote computing system 900 further includes a housing 908 and a support member 910A. The base plate 906 is connected to the housing 908 through the support member 910A. The housing 908 further includes output device(s) 912 and input device(s) 914. Preferably, the remote computing system 900 is integrated as a single unit with the support member coupling the base plate 906 and the housing 908, thus providing a unit in a one-piece construction.

It will be appreciated that other physiological transducing devices can be utilized in addition to the electronic scale 902. For example, a blood pressure measurement apparatus and an electrocardiogram (EKG) measurement apparatus can be utilized with the remote computing system 900 for recordation and/or transmission of blood pressure and EKG measurements to a remote location. In addition, a glucometer can be utilized with the remote computing system 900 for measuring the glucose level in the patient's blood. It will be appreciated that other monitoring devices of physiological body functions that provide an analog or digital electronic output can be utilized with the remote computing system 900, and are connected to the appropriate functional units as shown above in FIGS. 5-7.

Referring to FIGS. 9B, 9C, 9D and 9E it will be appreciated that the support member 910A (FIG. 1A) can be made adjustable. For example, FIG. 9B illustrates an embodiment of the present disclosure that utilizes a telescoping support member 910B. Likewise, FIG. 9C illustrates an embodiment of the remote computing system 900 that utilizes a folding articulated support member 910C. FIG. 9D illustrates yet another embodiment of the present disclosure utilizing support member 910D that folds at a pivot point 914 located at its base.

It will also be appreciated that other types of articulated and folding support members can be utilized in other embodiments of the present disclosure. For example, FIG. 9E illustrates an embodiment of the present disclosure that provides a support member 910E that is removably insertable into a socket 916. A cable 918 is passed through the support member 910E to carry electrical signals from the electronic scale 902 to the housing 908 for further processing. A tether 920 is provided to restrain the movement of the support member 910E relative to the base plate 906 once it is removed from the socket 916.

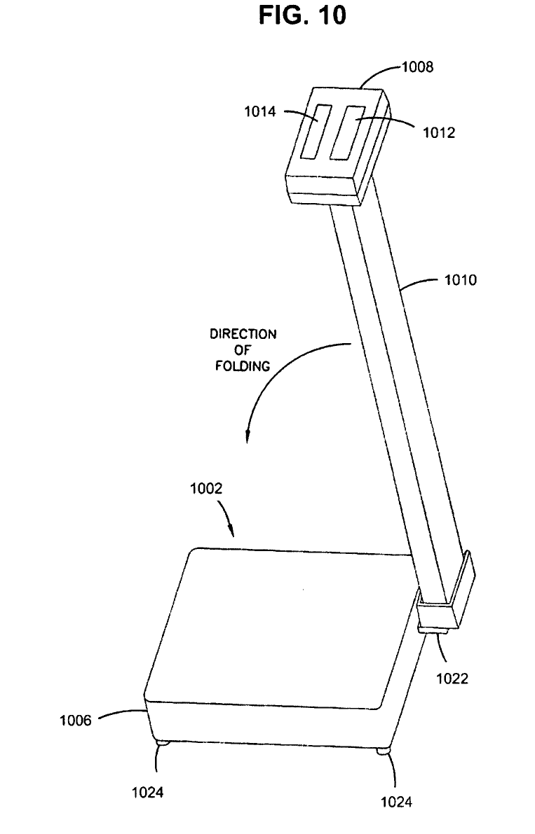

Referring now to FIG. 10, the structure of a remote computing system 1000 is illustrated according to one embodiment of the present disclosure where the support member 1010 folds about pivot point 1022. Folding the integrated monitoring apparatus about pivot point 1022 provides a convenient method of shipping, transporting or moving the apparatus in a substantially horizontal orientation. The preferred direction of folding is indicated in the illustration, however, the support member 1010 can be made to fold in either direction. Furthermore, an embodiment of the present disclosure provides rubber feet 1024 underneath the base plate 1006 of the scale 1002.

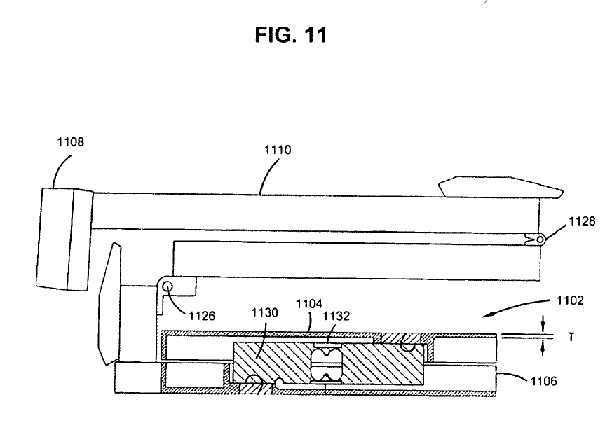

Referring now to FIG. 11, the structure of a remote computing system 1100 is illustrated according to one embodiment of the present disclosure that provides an articulated, folding support member 1110. The support member 1110 folds at two hinged pivot points 1126, 1128. Also illustrated is a sectional view of a scale 1102, top plate 1104, base plate 1106, load cell 1130 and strain gage 1132.

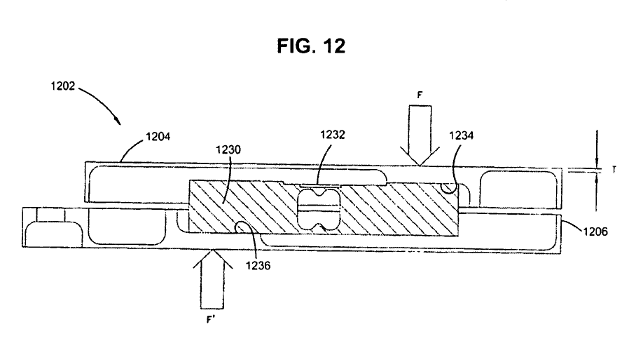

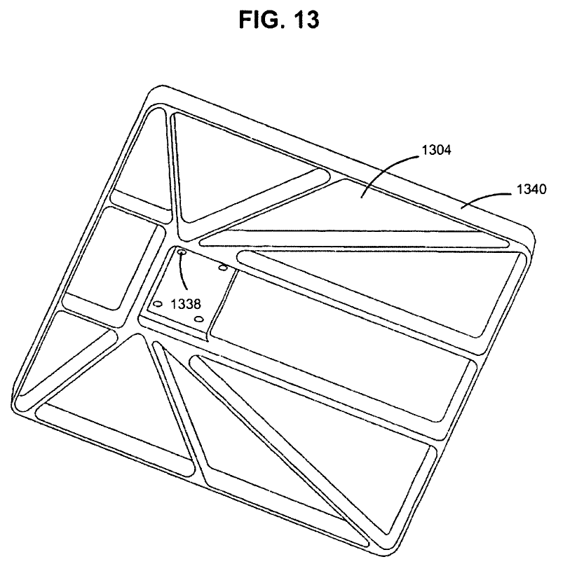

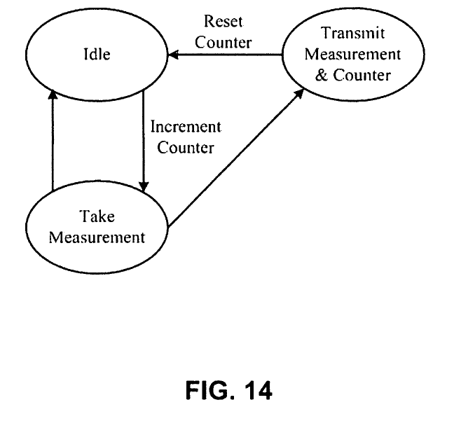

Referring now to FIG. 12, a sectional view of a scale portion of a remote computing system 1200 is shown according to one embodiment of the present disclosure. The scale 1202 comprises a top plate 1204 and a base plate 1206. The top plate 1204 and the base plate 1206 having a thickness “T”. A load cell 1230 is disposed between the top plate 1204 and the base plate 1206 and rests on support/mounting surfaces 1234 and 1236.

The load cell 1230 is a transducer that responds to forces applied to it. During operation, when a patient steps on the electronic scale 1202, the load cell 1230 responds to a force “F” transmitted through the top plate 1204 and a first support/mounting surface 1234. The support/mounting surface 1234 is in contact with a first end on a top side of the load cell 1230. A force “F′” that is equal and opposite to “F” is transmitted from the surface that the electronic scale 1202 is resting on, thorough the base plate 1206 and a second support/mounting surface 1236. The second support/mounting surface 1236 is in contact with a second end on a bottom side of the load cell 1230. In one embodiment, the load cell 1230 is attached to the top plate 1204 and the base plate 1206, respectively, with bolts that engage threaded holes provided in the load cell 1230. In one embodiment the load cell 1230 further comprises a strain gage 1232.

The strain gage 1232 is made from ultra-thin heat-treated metallic foils. The strain gage 1232 changes electrical resistance when it is stressed, e.g. placed in tension or compression. The strain gage 1232 is mounted or cemented to the load cell 1230 using generally known techniques in the art, for example with specially formulated adhesives, urethanes, epoxies or rubber latex. The positioning of the strain gage 1232 will generally have some measurable effect on overall performance of the load cell 1230. Furthermore, it will be appreciated by those skilled in the art that additional reference strain gages can be disposed on the load cell where they will not be subjected to stresses or loads for purposes of temperature compensating the strain gage 1232 under load. During operation over varying ambient temperatures, signals from the reference strain gages can be added or subtracted to the measurement signal of the strain gage 1232 under load to compensate for any adverse effects of ambient temperature on the accuracy of the strain gage 1232.

The forces, F and F′, apply stress to the surface on which the strain gage 1232 is attached. The weight of the patient applies a load on the top plate 1204. Under the load the strain gage(s) 1232 mounted to the top of the load cell 1230 will be in tension/compression as the load cell bends. As the strain gage 1232 is stretched or compressed its resistance changes proportionally to the applied load. The strain gage 1232 is electrically connected such that when an input voltage or current is applied to the strain gage 1232, an output current or voltage signal is generated that is proportional to the force applied to the load cell 1230. This output signal is then converted to a digital signal by an A/D converter, such as those described above.

The design of the load cell 1230 having a first end on a top side attached to the top plate 1204 and a second end on a bottom side attached to the base plate 1206 provides a structure for stressing the strain gage 1232 in a repeatable manner. The structure enables a more accurate and repeatable weight measurement. This weight measurement is repeatable whether the scale 1202 rests on a rigid tile floor or on a carpeted floor.

Referring now to FIG. 13 illustrates one embodiment of the top plate 1304 that provides four mounting holes 1338 for attaching the base plate to one end of the load cell. The base plate provides similar holes for attaching to the other end of the load cell. The top plate and the base plate (not shown) each comprise a plurality of stiffening ribs 1340 that add strength and rigidity to the electronic scale.

Table 1 shows multiple comparative weight measurements taken with an electronic scale resting on a tile floor and a carpeted floor without rubber feet on the scale. The measurements were taken using the same load cell. The thickness “T” of the top plate and supporting ribs was 0.125″ except around the load cell, where the thickness of the supporting ribs was 0.250″. The thickness of the load cell support/mounting surfaces 96, 98 (FIG. 9) was 0.375″. As indicated in Table 1, with the scale resting on a tile floor, the average measured weight was 146.77 lbs., with a standard deviation of 0.11595. Subsequently, with the scale resting on a 0.5″ carpet with 0.38″ pad underneath and an additional 0.5″ rug on top of the carpet, the average measured weight was 146.72 lbs., with a standard deviation of 0.16866.

TABLE 1
Thick Scale Parts Around Load Cell 0.250″

| TILE (lbs.) | CARPET (lbs.) |
|---|---|
| 146.9 | 146.7 |
| 146.7 | 147 |
| 146.9 | 146.6 |
| 146.8 | 146.7 |
| 146.6 | 146.6 |
| 146.8 | 147 |
| 146.8 | 146.5 |
| 146.7 | 146.6 |
| 146.9 | 146.8 |
| 146.6 | 146.7 |
| 0.11595 (stddev) | 0.16866 (stddev) |
| 146.77 (average) | 146.72 (average) |

Table 2 shows multiple weight measurements taken with the scale on a tile floor and a carpeted floor with rubber feet on the bottom of the scale. The measurements were taken using the same load cell. The thickness “T” of the top plate was 0.125″ including the thickness around the load cell. As indicated in Table 2, with the scale resting on a tile floor on rubber feet, the average measured weight was 146.62 lbs., with a standard deviation of 0.07888. Subsequently, with the scale resting on a 0.5″ carpet with 0.38″ pad underneath and an additional 0.5″ rug on top of the carpet, the average measured weight was 146.62 lbs., with a standard deviation of 0.04216.

TABLE 2
Thin Scale Parts Throughout 0.125″

| TILE (lbs.) | CARPET (lbs.) |
|---|---|
| 146.7 | 146.7 |
| 146.7 | 146.7 |
| 146.6 | 146.6 |
| 146.6 | 146.6 |
| 146.6 | 146.6 |
| 146.6 | 146.6 |
| 146.5 | 146.6 |
| 146.7 | 146.6 |
| 146.5 | 146.6 |
| 146.7 | 146.6 |
| 0.07888 (stddev) | 0.04216 (stddev) |
| 146.62 (average) | 146.62 (average) |

Table 3 shows multiple weight measurements taken with an off-the-shelf conventional electronic scale. As indicated in Table 3, with the off-the-shelf conventional scale resting on the tile floor, the average measured weight was 165.5571 lbs., with a standard deviation of 0.20702. Subsequently, with the off-the-shelf conventional scale resting on a 0.5″ carpet with 0.38″ pad underneath and an additional 0.5″ rug on top of the carpet, the average measured weight was 163.5143 lbs., with a standard deviation of 0.13093.

TABLE 3
Off-The-Shelf Conventional Scale

| TILE (lbs.) | CARPET (lbs.) |
|---|---|
| 165.9 | 163.5 |
| 165.5 | 163.4 |
| 165.8 | 163.7 |
| 165.4 | 163.6 |
| 165.5 | 163.6 |
| 165.4 | 163.5 |
| 165.4 | 163.3 |
| — | 163.4 |
| 0.20702 (stddev) | 0.13093 (stddev) |
| 165.5571 (average) | 163.5143 (average) |
| 2.042857 (% of difference) | 1.249345 (% of difference) |

The summary in Table 4 is a comparative illustration of the relative repeatability of each scale while resting either on a tile floor or on a carpeted floor.

TABLE 4
SUMMARY OF DATA:

| | | | | | |
|---|---|---|---|---|---|
| | | | | | TILE VS. |
| TRIAL | TILE | STDDEV | CARPET | STDDEV | CARPET |

Heavy Scale Parts All 0.125″ Except Cell Around the Load Cell 0.250″

| | | | | | |
|---|---|---|---|---|---|
| 1 | 146.77 | 0.1159 | 146.72 | 0.1686 | 0.05 |
| 2 | 146.67 | 0.0823 | 146.72 | 0.1906 | 0.05 |

Thin Scale Parts All 0.125″

| | | | | | |
|---|---|---|---|---|---|
| 1 | 146.62 | 0.0788 | 146.62 | 0.04216 | 0.00 |

Off-The-Shelf Conventional Scale

| | | | | | |
|---|---|---|---|---|---|
| 1 | 165.55 | 0.207 | 163.51 | 0.1309 | 2.04 |

The foregoing description was intended to provide a general description of the overall structure of several embodiments of the present disclosure, along with a brief description of the specific components of these embodiments of the present disclosure. The following provides examples of operation of the remote computing system.

In operating the remote computing system, an ambulatory patient utilizes the system to obtain a measurement of a particular physiological parameter. For example, an ambulatory patient suffering from chronic heart failure will generally be required to monitor his or her weight as part of in-home patient managing system. Accordingly, the patient measures his or her weight by stepping onto the electronic scale, integrally located within the base plate of the remote computing system. Alternately, the patient measures his or her glucose level by connecting a glucometer to the housing.

In some embodiments the communication device of the remote computing system will only activate if the measured weight or other physiological parameter is within a defined range such as +/−10 lbs, +/−10% or any selected predetermined value of a previous measurement. The patient's previous symptom free parameter is stored in a memory. This prevents false activation of the communication device if a child, pet, or other person accidentally steps onto the electronic scale.

Upon measuring the weight or other physiological parameter, the system determines whether it is within a defined, required range such as +/−10 lbs. or +/−10% of a previously recorded weight stored in memory. The remote computing system then initiates a call via the communication device to the remote site. Communication is established between the remote computing system and the local computing system. In one embodiment of the present disclosure, the patient's weight is electronically transferred from the remote computing system at the remote site to the local computing system at the local site. At the local site a computer program compares the patient's weight with the dry weight and wellness information and updates various user screens. The program can also analyze the patient's weight trend over the previous 1-21 days. If significant symptoms and/or excessive weight changes are reported, the local computing system alerts the medical care provider who can provoke a change to the patient's medication dosage, or establish further communication with the patient such as placing a telephone to the patient. The communication between the patient's remote location and the local location can be one way or two way communication depending on the particular situation.

To establish the patient's overall condition, the patient is prompted via the output device(s) to answer questions regarding various wellness parameters. An exemplary list of questions, symptoms monitored and the related numerical score is provided in Table 5 as follows:

TABLE 5
Health Check Score

| Question | Symptom | Value |
|---|---|---|
| Above Dry Weight? | Fluid accumulation | 10 |
| Are you feeling short of breath? | Dyspnea | 10 |
| Did you awaken during the night short | Paroxysmal nocturnal | 5 |
| of breath? | dyspnea | |
| Did you need extra pillows last night? | Congestion in the lungs | 5 |
| Are you coughing more than usual? | Congestion in the lungs | 3 |
| Are your ankles or feet swollen? | Pedal edema | 5 |
| Does your stomach feel bloated? | Stomach edema | 3 |
| Do you feel dizzy or lightheaded? | Hypotension | 5 |
| Are you more tired than usual? | Fatigue | 2 |
| Are you taking your medication? | Medication compliance | 7 |
| Has your appetite decreased? | Appetite | 2 |
| Are you reducing your salt intake? | Sodium intake | 1 |
| Did you exercise today? | Fitness | 1 |

At the local site the medical professional caregiver evaluates the overall score according to the wellness parameter interrogation responses (as shown in Table 5). For example, if the patient's total score is equal to or greater than 10, an exception is issued and will either prompt an intervention by the medical professional caregiver in administering medication, or prompt taking further action in the medical care of the patient.

Upon uploading the information to the local computing system, the medical professional caregiver may telephone the patient to discuss, clarify or validate any particular wellness parameter or physiological data point. Furthermore, the medical professional caregiver may update the list of wellness parameter questions listed in Table 5 from the local site over the two-way communication network. Modifications are transmitted from the local computing system via communication device, over the communication network, through communication device and to the remote computing system. The modified query list is then stored in the memory of the microprocessor system.

Similar to the preceding example of weight management, glucose levels of a person with diabetes can be monitored. The apparatus can include a glucose meter. The person can insert a test strip, having a small amount of blood, into the glucose meter. The glucose meter can measure the glucose level in the blood and transmit that information through the communication device over the communication network to the communication device and the local computing system. The local computing system can track and monitor the glucose levels of the person. If the glucose level is abnormal, a caregiver and/or patient can be notified.

FIG. 14 depicts a state transition diagram of another embodiment of the scheme of FIG. 1. According to the embodiment of FIG. 14, a count parameter may be maintained by the device utilizing the exhaustible medical supply. For example, with reference to FIG. 3, the count may be maintained by the glucometer 304, or may be maintained by the patient monitoring device 302. Such an embodiment is useful when the device utilizing the expendable medical supply may be used one or more times between communication sessions with the remote computing system 208 (see FIG. 2) operated by the call center, health care facility, etc. The embodiment of FIG. 14 prevents such intersession usage from going unobserved and therefore uncounted. The method of FIG. 14 may be executed by either the device utilizing the disposable medical supply or by any device that communicates therewith (e.g., the patient monitoring device 302). For the sake of illustration only, the method of FIG. 14 is described as though it is being executed by the patient monitoring device 302 with a glucometer coupled thereto.

As can be seen from FIG. 14, in between uses of the patient monitoring device and/or the glucometer are in an idle state 1400. Upon command, the glucometer transitions to a measurement state 1402 wherein it develops a blood glucose measurement based upon a blood sample delivered on a disposable strip. During this process, a test strip and a lance may be expended, for example. Thus, a counter corresponding to each expendable/exhaustible/disposable item is increment (e.g., a counter corresponding to the test strip is incremented, and a counter corresponding to the lance is incremented).

In the context of an inter-session measurement, the glucometer and patient monitoring device return to the idle state 1400. Thereafter, the glucometer may be commanded to take another measurement, whereupon transition to state 1402 will again occur, and the aforementioned counters are again incremented. When the blood measurement is obtained as a part of a patient monitoring session, a transition to the transmit glucose measurement and counters state 1404 occurs (of course, the patient monitoring device may pose questions to the patient, as described previously, prior to such transition). During execution of state 1404, the patient monitoring device transmits both the blood glucose level and the aforementioned counters to the remote computing system 208.

The remote computing system 208 responds by executing the method of FIG. 1, with the following exception. Instead of incrementing the supply counter by one in operation 108, the supply counter is incremented by the corresponding counter value received from the patient monitoring device. (Example: assuming that the patient has measured his blood glucose level ten times since the last communication with the remote computing system 208, supply counter X is incremented by ten, i.e., X=X+10, indicating that ten test strips have been expended, and/or that ten lances have been expended.)

According to some embodiments, Y is a function of the purchased expendable medical supply. For example, assuming that a supply of 100 test strips is purchased, the remote computing system may be programmed to set Y equal to 90 (e.g., Y=0.9*the number of test strips purchased). On the other hand, assuming that a supply of 200 test strips is purchased, the remote computing system may be programmed to set Y equal to 180 (e.g., Y=0.9*the number of test strips purchased).

The logical operations of the various embodiments of the present disclosure can be implemented as a sequence of computer implemented steps running on a computing system. The implementation is a matter of choice dependent on the performance requirements of the computing system implementing the disclosure. The invention can be implemented as a computer process, a computing system, or as an article of manufacture such as a computer program or computer readable media. The computer program product can be a computer storage media readable by a computer system and encoding a computer program of instructions for executing a computer process. The computer program product can also be a propagated signal on a carrier readable by a computing system and encoding a computer program of instructions for executing a computer process.

Thus, it will be appreciated that the previously described embodiments provide a method and system for the tracking and monitoring or medical supplies and the automatic reordering of such supplies.

Also, it will be appreciated that the previously described embodiments provide many advantages, including addressing the needs in the medical profession for an apparatus and method capable of monitoring and transmitting physiological and wellness parameters of ambulatory patients to a remote site whereby a medical professional caregiver can evaluate such physiological and wellness parameters and make decisions regarding the patient's treatment.

Also, it will be appreciated that the previously described embodiments provide other advantages, including addressing the need for an apparatus for monitoring and transmitting such physiological and wellness parameters that is available in an easy to use portable integrated single unit.

Furthermore, it will be appreciated that the previously described embodiments provide still other advantages, including addressing the need for medical professional caregivers to monitor and manage the patient's condition to prevent the rehospitalization of the patient, and to prevent the patient's condition from deteriorating to the point where hospitalization may be required.

The above specification, examples and data provide a complete description of the manufacture and use of the composition of the invention. Since many embodiments of the invention can be made without departing from the spirit and scope of the invention, the invention resides in the claims hereinafter appended.

The claimed invention is:

1. A computerized method for determining need for additional medical supplies, the method comprising:
receiving a test result from a remote computing device;
updating a supply counter based on receiving the test result;
determining if the supply counter exceeds a limit; and
triggering a process to reorder supplies when the supply counter exceeds the limit.

2. The method of claim 1 wherein receiving a test result from a remote computing device comprises receiving a blood sugar test result from a remote computing device.

3. The method of claim 1 wherein triggering a process to reorder supplies comprises triggering a process to reorder glucose test strips.

4. The method of claim 1 wherein triggering a process to reorder supplies comprises triggering a process to reorder lancets.

5. The method of claim 1 wherein receiving a test result from a remote computing device comprises receiving a cholesterol test result from a remote computer device.

6. The method of claim 1 wherein triggering a process to reorder supplies comprises triggering a process to reorder at least one cholesterol blood test kit.

7. The method of claim 1 wherein triggering includes triggering a process to order a quantity of supplies greater than or equal to the limit.

8. The method of claim 1 further comprising setting the supply counter at an initial value.

9. The method of claim 1 wherein triggering is accomplished by a local computing device that is distant from the remote computing device.

10. The method of claim 1 wherein triggering prompts a user before reordering supplies.

11. The method of claim 1 wherein triggering directly initiates reordering supplies and a billing process.

12. The method of claim 1, further comprising receiving an increment value from the remote computing device, and wherein the act of updating the supply counter comprises adding the increment value to the supply counter.

13. The method of claim 1, wherein the limit is determined based at least upon a quantity of the supplies that are reordered.

14. A system for determining if additional medical supplies are necessary, the system comprising:
a receive module that receives a test result from a remote computing device;
an update module that updates a supply counter based on receiving the test result;
a determination module that determines if the supply counter exceeds a limit; and
a trigger module that if the supply counter exceeds the limit, triggers a process to reorder the supplies.

15. The system of claim 14 further comprising a set module that sets the supply counter at an initial value.

16. The system of claim 14 wherein the receive module receives a glucose blood test result.

17. The system of claim 14 wherein the trigger module triggers a process to reorder glucose test strips.

18. The system of claim 14 wherein the receive module receives a cholesterol test result.

19. The system of claim 14 wherein the trigger module triggers a process to reorder a cholesterol test.

20. The system of claim 14 wherein the trigger module prompts an ambulatory patient using the remote computing device prior to reordering supplies.

21. The system of claim 14 wherein the update module comprises an up-counter, a down-counter, or an up/down counter.

22. The system of claim 14 wherein the system is distant from the remote computing system.

23. The system of claim 14, wherein the receive module further receives an increment value from the remote computing device, and wherein the update module updates the supply counter by adding the increment value to the supply counter.

24. The system of claim 14, further comprising a limit establishment module that determines the limit based at least upon a quantity of the supplies that are reordered.

25. A system for remotely monitoring glucose levels in an ambulatory patient, the system comprising:
a remote computing system that tests the glucose level of the ambulatory patient, the remote computing system including a communication device connected to a communication network;
a local computing system that includes a communication device connected to the communication network, the local computing system receiving the glucose level from the remote computing system through the communication network;
wherein the local computing system tracks the glucose level of the ambulatory patient, and if the glucose level is outside certain parameters, the local computing system alerts a caregiver such that the caregiver can contact the ambulatory patient.

26. The system of claim 25 wherein the local computing system updates a supply counter upon receiving the glucose level, determines if the supply counter exceeds a limit, and automatically triggers a process to reorder glucose test strips if the supply counter exceeds the limit.

27. The system of claim 27 wherein the local computing system tracks testing regularity of the ambulatory patient.

28. The system of claim 27 wherein if the glucose level is outside certain parameters, the local computing system alerts the ambulatory patient.

29. The system of claim 27 wherein the local system is in two-way communication with the remote computing system such that the caregiver can send and receive messages from the ambulatory patient.

30. A system for remotely monitoring a physiological parameter of an ambulatory patient, the system comprising:
a remote computing system that determines the physiological parameter of the ambulatory patient using single-use medical supplies, the remote computing system including a communication device connected to a communication network; and
a local computing system that includes a communication device connected to the communication network,
wherein the local computing system tracks the physiological parameter of the ambulatory patient,
and if the physiological parameter is outside certain parameters, the local computing system alerts a caregiver such that the caregiver can contact the ambulatory patient.

31. The system of claim 30 wherein the local computing system updates a supply counter upon receiving the physiological parameter, determines if a supply counter exceeds a limit, and automatically triggers a process to reorder the single-use medical supplies if the supply counter exceeds the limit.

32. The system of claim 30 wherein if the system automatically triggers a process to reorder the single-use medical supplies, the system prompts the ambulatory patient.

33. The system of claim 30 wherein the local computing system tracks testing regularity of the ambulatory patient.

34. The system of claim 30 wherein the single use medical supplies are glucose test strips.

35. The system of claim 30 wherein if the physiological parameter is outsider certain parameters, the local computing system alerts the ambulatory patient.
